(19) United States
(12) Patent Application Publication
(10) Pub. No.: US 2012/0089231 A1
(43) Pub. Date: Apr. 12, 2012

(54) INTERVERTEBRAL IMPLANT AND TOOL FOR PLACING SAME

(75) Inventor: Philippe Prestigiacomo, Lacanau-Ocean (FR)

(73) Assignee: CREASPINE, Pessac (FR)

(21) Appl. No.: 13/375,790

(22) PCT Filed: May 21, 2010
(86) PCT No.: PCT/FR2010/050995

§ 371 Date: Dec. 2, 2011

(30) Foreign Application Priority Data
Jun. 10, 2009 (FR) 09/02808

(51) Int. Cl. A61F 2/44

(52) U.S. Cl. 623/17.16

(57) ABSTRACT

The invention relates to an intervertebral implant (1) designed to be placed in an intervertebral space by 90° pivoting and without preliminary spacing of the vertebrae, that comprises two bearing surfaces (7a, 7b) adapted for interacting with the lower or upper portions of the vertebral bodies on the one hand, and two side surfaces (3a, 3b) substantially perpendicular to the bearing surfaces (7a, 7b) on the other hand. The implant is characterized in that it is elastically deformable so as to be deformed from a placing configuration in which the two side surfaces (3a, 3b) are close to each other into a service configuration in which the two side surfaces (3a, 3b) are spaced apart from each other, and in that it comprises a threaded opening (17) capable of interacting with a tool for pivoting the implant by 90° and troughs (15a, 15b) formed lengthwise in the side surfaces (3a, 3b) and adapted for receiving the blades of said tool for moving the implant from one configuration to the other.

### TECHNICAL FIELD

The invention relates in particular to an intervertebral implant as well as a tool for placing said implant in the intervertebral space.

### BRIEF DESCRIPTION OF RELATED ART

When an intervertebral disk is damaged following a compression or because of degeneration, like those frequently encountered in pathologies related to herniated disks, it is necessary to stabilize the disk to eliminate the pain felt by the patient. Most often, the chosen solution consists of making the two vertebra in question integral, for example using intervertebral implants and bone grafts: this is called bone fusion of those vertebrae.

Intervertebral implants are commonly used in bone surgery of the vertebral column; most often, the vertebrae are spaced apart using distractors to insert and position said implants, as explained in particular in patent documents U.S. Pat. No. 6,942,698, FR2767676 and WO99/38463. However, the use of such distractors, which must be placed carefully, complicates the surgical operation and is highly invasive.

Also known from patent application US02/0029082 is the use of an implant whereof the placement is done without a distractor, owing to an insertion of the primary piece between the vertebral bodies followed by a 90° rotation. However, the piece used has a contact surface with the two vertebrae that is too reduced to be able to guarantee a stable positioning of the prosthesis, without any risk of it tilting and causing unwanted effects for the patient.

One thus uses an additional piece, a stabilizer, acting like a support that prevents tilting of the implant. However, the use of such an implant with several parts complicates the medical placement operation between the vertebrae, as it in particular requires the use of additional fastening means to keep the main piece assembled with the additional piece, or in that it requires an additional step for filling the additional piece with a filler.

### BRIEF SUMMARY

The present invention aims to resolve avoid all or part of the aforementioned drawbacks.

To that end, the present invention relates, according to a first aspect, to an intervertebral implant designed to be placed in an intervertebral space by 90° pivoting and without preliminary spacing of the vertebrae, comprising two bearing surfaces adapted for interacting with the lower or upper portions of the vertebral bodies on the one hand, and two side surfaces substantially perpendicular to the bearing surfaces on the other hand, the implant being remarkable in that:
it is elastically deformable so as to be deformed from a placing configuration in which the two side surfaces are close to each other into a service configuration in which the two side surfaces are spaced apart from each other, and in that
it comprises a threaded opening capable of interacting with a tool for pivoting the implant by 90° and troughs formed lengthwise in the side surfaces and adapted for receiving the blades of said tool for moving the implant from one configuration to the other.

Such an implant is easy to use and inexpensive, given that it can be made in a single piece and does not require the use of additional outside pieces to maintain it in the intervertebral space.

Once placed, the implant has great stability owing to the spacing of its side surfaces, under the effect of its elasticity alone.

Such an implant allows the operator to avoid, in addition to the steps for inserting the implant into the patient's body, having to finalize the implant placement operation either through steps for fastening various pieces, or through liquid injection steps. Such an implant consequently limits the medical complications due to the handling and insertion of various foreign bodies into the intervertebral space and near the nervous structures.

The presence of the threaded opening makes it possible to engage the implant with the tool owing to which it is possible to pivot said tool, and the presence of the trough allows the passage of the blades of said tool, which prevent the two bearing surfaces from being spaced apart from one another as long as the pivoting has not been done.

The maintenance of these two bearing surfaces against one another before pivoting of the implant makes it possible to insert that implant between the adjacent vertebral bodies without over-distraction of those bodies.

This is even more true inasmuch as the troughs formed on the side surfaces of the implant make it possible to place blades of the tool without the latter protruding from the volume of the implant, i.e. without additional bulk, thereby contributing to the lack of need for over-distraction.

According to other optional features of the implant according to the invention:
said bearing surfaces include slots: this makes it possible to retain the implant by friction between the two vertebral bodies;
said side surfaces are smooth, outside the zone of said troughs: this makes it possible to slide the implant easily between the two adjacent vertebral bodies before pivoting it;
the junction zones between said bearing surfaces and said side surfaces are rounded: this makes it possible to facilitate pivoting of the implant under the effect of the tool;
said side surfaces are associated with elastic branches connected to one another by a bridge;
said implant comprises troughs formed in the direction of the length of said side surfaces, and a threaded opening pierced in said bridge;
preferably, the side surfaces can pass into the spaced apart configuration by pivoting said elastic branches around an axis passing through said bridge;
preferably, said side surfaces are substantially in the shape of an isosceles trapezoid whereof the side with the shortest length belongs to said bridge: such a morphology of the implant is particularly adapted to the shape of the intervertebral space in certain zones of the vertebral column;
preferably, the relative incline between said bearing surfaces is typically 10-25°: such an angular offset between the non-parallel opposite sides two by two of the isosceles trapezoid best respects the geometry of the intervertebral spaces in the lumbar zone of the vertebral column.

According to a second aspect, the present invention relates to a tool for placing an intervertebral implant as previously described, remarkable in that it comprises:
a sleeve;
a rod able to slide in said sleeve comprising a smooth portion and, at its end, a screw pitch provided to interact with said threaded opening;
two strips fixed opposite one another on said sleeve and provided to interact with said troughs.

### BRIEF DESCRIPTION OF THE DRAWINGS

The present invention will be better understood upon reading the following detailed description, illustrated by the appended figures, in which:

FIGS. 1a, 1b, 1c and 1d respectively show a perspective view, a side view on the front surface, a side view on a side surface and a top view of an implant in the service configuration according to the present invention, provided to be placed preferably through the posterior route in the vertebral column;

FIGS. 2 to 6 show a perspective view of the implant illustrated in FIGS. 1a, 1b, 1c and 1d with its placement tool, during the different stages of positioning the tool on the implant;

FIGS. 7 and 8 show a side view of two adjacent vertebrae during the steps for placing an implant as illustrated in FIGS. 1a, 1b, 1c and 1d, in the intervertebral space;

FIGS. 9 to 12 show a top view of one or two implants, as illustrated in FIGS. 1a, 1b, 1c and 1d, during steps for placement in an intervertebral space, FIGS. 8 and 9 showing two different views of the implant just after it is pivoted 90° in the vertebral column;

FIGS. 13a, 13b on the one hand, and 14a, 14b on the other hand show, in perspective and top view, a view of another embodiment of an implant according to the present invention, provided to be installed preferably laterally in the vertebral column, in the placing configuration and service configuration, respectively; and

FIG. 15 shows a top view of the implant illustrated in FIGS. 14a and 14b in the service configuration in an intervertebral space.

### DETAILED DESCRIPTION

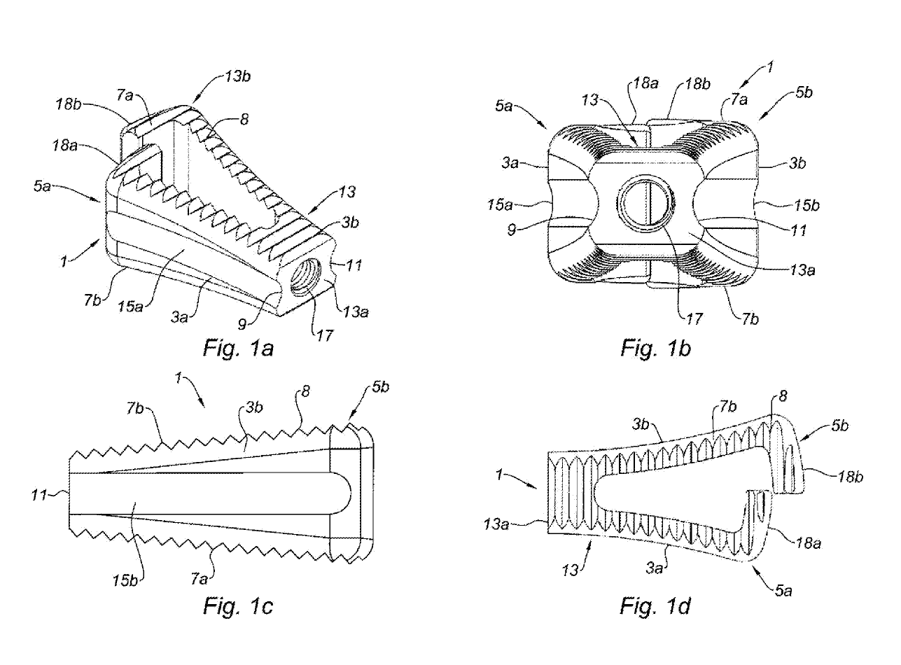

The intervertebral implant 1 shown in FIGS. 1a, 1b, 1c and 1d is designed to be implanted preferably between lumbar vertebrae of a patient, through the posterior route. This implant 1 comprises side surfaces 3a and 3b respectively belonging to two branches 5a and 5b. This side surfaces are substantially in the shape of an isosceles trapezoid whereof the sides of equal lengths are adjacent to the bearing surfaces 7a and 7b striated by slots 8. The sides 9 and 11 that are shorter than the side surfaces 3a and 3b, respectively, partially delimit a surface 13a of a bridge 13 joining the two side surfaces 3a and 3b. The implant 1 also comprises troughs 15a and 15b formed on the side surfaces 3a and 3b; outside these troughs, the side surfaces 3a and 3b are preferably smooth. The bridge 13 is perforated by a threaded opening 17. The branches 5a and 5b have different lengths and are provided with extensions 18a and 18b that close the space between said branches.

Preferably, as shown in particular in FIG. 1b, the connecting zones between the side surfaces 3a and 3b and the bearing surfaces 7a and 7b are preferably rounded.

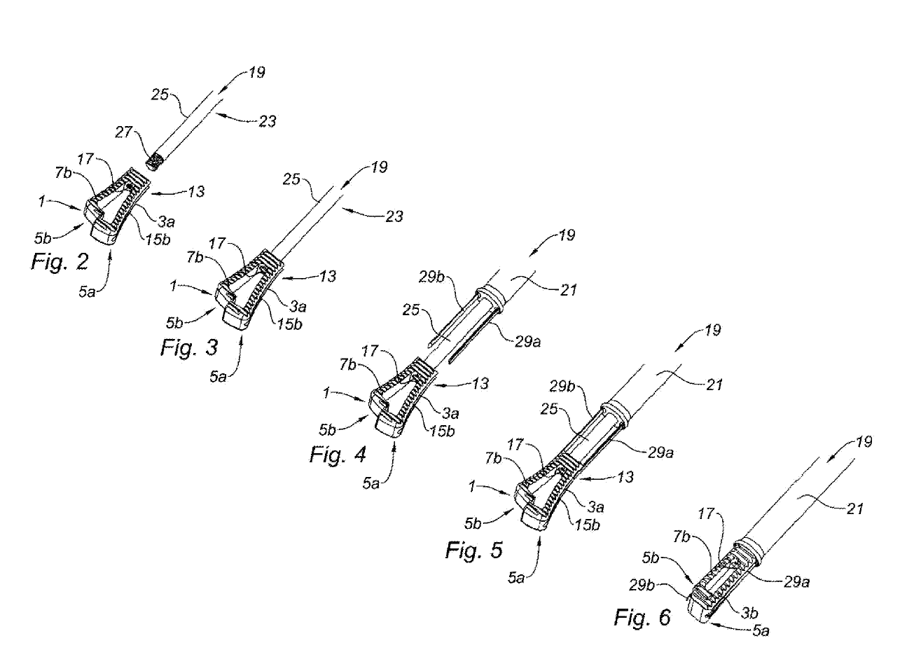

Before the placement of the implant 1 in the vertebral column of the patient, the operator positions a placement tool 19 on the implant 1. Such a tool is shown in particular in FIGS. 2 to 6: it comprises a sleeve 21 in which a rod 23, comprising a smooth end 25, is mounted slidingly pivotably; said rod 23 ends with a screw pitch 27, visible in FIG. 2.

The sleeve 21 is also equipped with two blades 29a and 29b placed on either side of the rod 23. At its end (not shown) opposite that having the screw pitch 27, the rod 23 comes out of the sleeve 21 and is provided with a gripping means making it possible to rotate and/or translate said rod relative to the sleeve.

FIGS. 2 and 3 show the screwing, by the operator, of the screw pitch 27 into the threaded opening 17 of the implant 1. The operator then slides the sleeve 21 onto the rod 23, as shown in FIGS. 4 and 5. Then, by applying slight pressure on the sleeve 21 while firmly maintaining the implant 1 via the rod 23, he inserts the entire smooth portion 25 of the rod 23 into the sleeve 21, so that the blades 29a and 29b slide completely into the troughs 15a and 15b of the implant 1 and bring the branches 5a and 5b closer to one another. Such a change in configuration of the implant 1 is illustrated by the transition from FIG. 5 to FIG. 6. When the branches 5a and 5b are in such a position closer to one another, the implant 1 is said to be in the “placing” configuration.

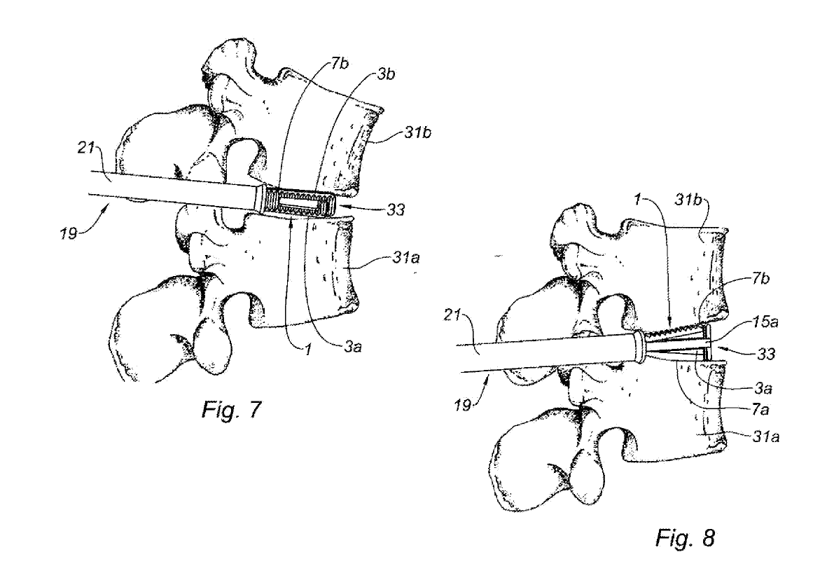

Once positioned as indicated above on the implant 1, the tool 19 is inserted through the posterior route at the vertebral column of a patient, as illustrated in FIGS. 7 to 12. The operator inserts the implant 1 in its placing configuration, so that the side surfaces 3a and 3b are placed opposite the lower 31a and upper 31b vertebrae (see FIG. 7).

This insertion by sliding the implant between the two adjacent vertebral bodies does not require any over-distraction, i.e. no spacing beyond the normal relative position of said two bodies, due to the fact on the one hand that the two branches 5a and 5b are kept against one another by the blades 29a and 29b of the tool 19, and on the other hand that these blades, positioned in the troughs 15a, 15b, do not protrude from the volume of the implant.

The sliding insertion of the implant is facilitated by the fact that its side surfaces 3a and 3b are smooth.

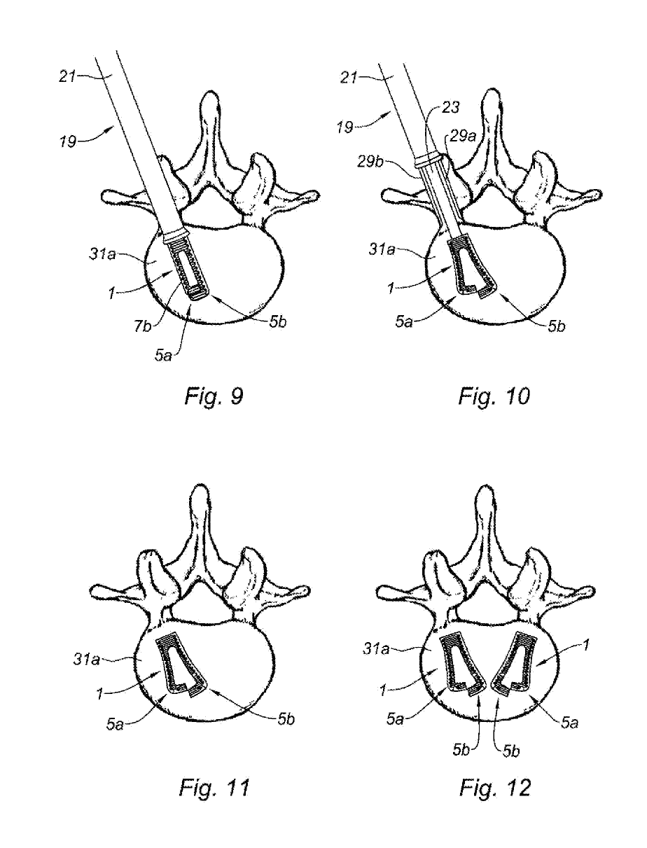

Secondly, as illustrated in FIGS. 8 and 9, the operator pivots the sleeve 21 so as to impart a 90° rotation to the implant, so that the bearing surfaces 7a and 7b come respectively across from the vertebrae 31a and 31b.

This pivoting operation is facilitated by the fact that the junction zones of the side surfaces 3a, 3b with the bearing surfaces 7a, 7b are rounded.

In order to cause the branches 5a and 5b to be spaced apart, the operator pulls on the sleeve 21 while keeping the rod 23 immobile, as shown in FIG. 10, which results in extracting the blades 29a and 29b from the troughs 15a and 15b and drives the spontaneous spacing apart of the branches 5a and 5b relative to one another, due solely to the elasticity of the material forming the implant.

The implant 1 in such a configuration is then said to be in the “service configuration.” The contact surface between the implant and the upper 31b and lower 31a vertebrae is therefore larger and allows better stability of the implant 1 in the intervertebral space 33.

The transition from the placing configuration to the service configuration is done under the sole elasticity of the material forming the implant 1, this material being able to be metal (e.g. titanium), polymer (e.g. PEEK—poly-ether-etherketone), composite or shape memory.

The slots 8, intended to maintain the implants 1 by friction in the intervertebral space 33, prevent any later displacement of the implant after it is positioned by the operator.

Lastly, the operator unscrews the screw pitch 27 from the threaded opening 17 by rotating the rod 23 in the sleeve 21 and leaves the implant 1 in position in the vertebral column of the patient in its service configuration, as shown in FIG. 11. It is also possible in this way to introduce two implants 1 into the vertebral column, as illustrated in FIG. 12.

As previously stated, the implant 1 is particularly adapted to secure the vertebrae 31a and 31b in the lumbar zone of the vertebral column, because the geometry of the side surfaces 3a and 3b of the implant inserted through the posterior route, as shown in FIGS. 7 and 8, makes it possible to respect the curvature of the vertebral column at that level. Ideally, the relative incline between the bearing surfaces 7a and 7b is comprised between 10 and 25°.

The present invention is not limited to the embodiment just described.

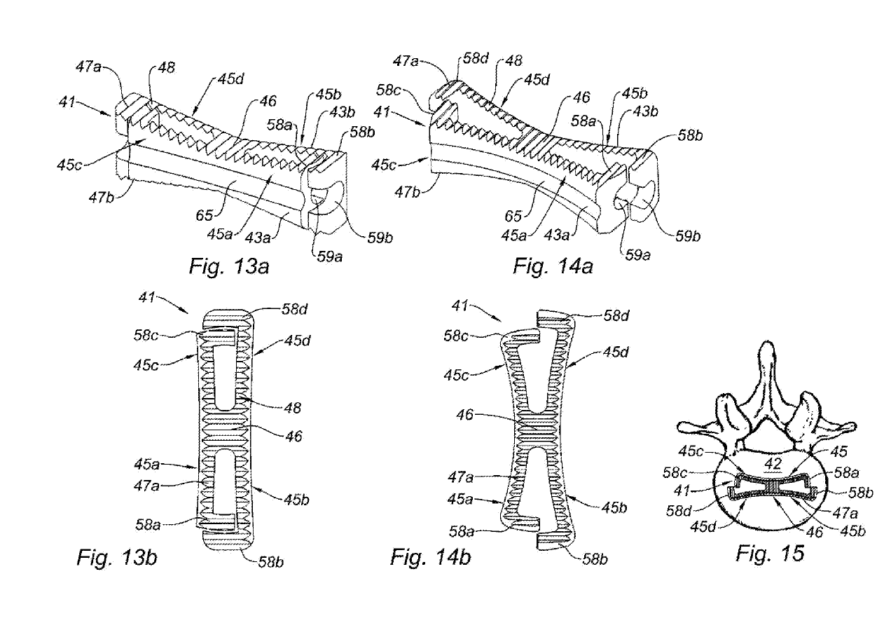

According to another embodiment 41 shown in FIGS. 13 to 15 adapted to be inserted into an intervertebral space 42, the implant can substantially assume the form of two implants 1 according to the preceding, placed end to end.

Such an implant 41 comprises side surfaces 43a and 43b each respectively belonging to two pairs of branches 45a, 45b and 45c, 45d with a beveled shape, arranged symmetrically relative to a bridge 46 connecting these two pairs of branches to one another.

Such an implant 41 can be obtained by associating two implants 1 each according to the embodiment described above, assembled by their bridges 13 using a threaded male piece interacting by screwing with the openings 17 of the bridges 13; this implant 41 can also be formed in a single piece.

It is also possible to provide that the bridge 46 cannot be centered at the middle of the pairs of branches 45a, 45b and 45c, 45d.

The upper and lower surfaces of the branches 45a, 45b, 45c, 45d form two bearing surfaces 47a and 47b each comprising a set of slots 48. The branches 45a and 45b are provided with extensions 58a and 58b, and the branches 45c and 45d are provided with extensions 58c and 58d comprising openings 59a and 59b intended to allow the insertion of the rod 23 of the tool 19 between said branches. The implant 1 also comprises troughs hollowed over the entire length of the side surfaces 43a and 43b, such as the trough 65, visible in FIGS. 13a and 14a, formed in the side surfaces 43a.

The implant 41 is appropriate, preferably, for placement and positioning in the vertebral column in an intervertebral space 42 of the thoracic region, and in particular through a lateral route, as shown in FIG. 15.

Of course, the present invention is in no way limited to the embodiments described and shown, provided as mere examples; and in particular concerning the method of inserting the implants according to the invention, one skilled in the art knows how to modify the morphology of the implant to facilitate its insertion through the anterior route into the intervertebral space.

1. An intervertebral implant designed to be placed in an intervertebral space by 90° pivoting and without preliminary spacing of vertebrae, comprising:
two bearing surfaces adapted for interacting with lower or upper portions of vertebral bodies, and two side surfaces substantially perpendicular to the bearing surfaces;
wherein the implant is elastically deformable so as to be deformed from a placing configuration in which the two side surfaces are close to each other into a service configuration in which the two side surfaces are spaced apart from each other,
the implant further comprising a threaded opening capable of interacting with a tool for pivoting the implant by 90° and troughs formed lengthwise in the side surfaces and adapted for receiving blades of said tool for moving the implant from one configuration to the other.

2. The intervertebral implant according to claim 1, wherein said bearing surfaces include slots.

3. The intervertebral implant according to claim 1, wherein said side surfaces are smooth, outside zone of said troughs.

4. The intervertebral implant according to claim 1, wherein junction zones between said bearing surfaces and said side surfaces are rounded.

5. The intervertebral implant according to claim 1, wherein said side surfaces are associated with elastic branches connected to one another by a bridge comprising said threaded opening.

6. The intervertebral implant according to claim 5, wherein said side surfaces are substantially in a shape of an isosceles trapezoid whereof a side with a shortest length belongs to said bridge.

7. The intervertebral implant according to claim 6, wherein a relative incline between said bearing surfaces is typically 10-25°.

8. A tool for placing an intervertebral implant according to claim 1, comprising:
a sleeve;
a rod able to slide in said sleeve comprising a smooth portion and, at an end, a screw pitch provided to interact with said threaded opening;
two strips fixed opposite one another on said sleeve and provided to interact with said troughs.
